(19) United States
(12) Patent Application Publication
(10) Pub. No.: US 2013/0263867 A1
(43) Pub. Date: Oct. 10, 2013

(54) SURGICAL DRAPE

(71) Applicant: Michael J. Young, Chicago, IL (US)

(72) Inventor: Michael J. Young, Chicago, IL (US)

(21) Appl. No.: 13/438,962

(22) Filed: Apr. 4, 2012

(51) Int. Cl. A61B 19/08

(52) U.S. Cl. 128/854

(57) ABSTRACT

A surgical drape made of a sheet of surgical drape material, includes a frangible outline defined on the sheet that defines a removable area of the sheet that is separable from the sheet by tearing along the first frangible outline to define a first fenestration through the sheet. A frangible line extends from the frangible outline to an outer peripheral edge of the sheet and defines a pre-defined path of tearing along which the sheet may be torn from the removable area to the outer peripheral edge.

### FIELD OF THE INVENTION

The present invention relates generally to surgical drapes for use during surgical and/or other medical procedures.

### DESCRIPTION OF THE BACKGROUND

Surgical drapes are used in medical procedures, such as surgeries, to cover the skin surrounding an exposed surgical field on a patient. The surgical drape serves as a barrier to help maintain a sterile condition at the exposed surgical field.

In the past, surgical drapes were often formed by a doctor, nurse, or other practitioner at the site of the surgery using multiple sheets of sterilized or sanitized drape material. The practitioner typically taped or otherwise secured the individual pieces of drape material around the periphery of the surgical field. This conventional method of forming the surgical drape, however, typically wasted drape material, and proved inconvenient and time consuming to perform.

It is often necessary for the practitioner to remove the surgical drape from the patient with one hand. For example, it may be necessary for the practitioner to hold a sterile dressing against the patient with one hand while simultaneously removing the surgical drape with his or her other hand. This is particularly in effect when a single drape with a cut-out center-hole is used, often utilized for smaller opthalmic, plastic, or anesthetic applications. Often, a surgeon may have spent considerable time in placing a specific dressing or obtaining vascular access, that mandates protecting the surgical site and not removing his/her hold-down hand in order to remove the sterile drape at the conclusion of the procedure while maintaining his tubing connections/sterile dressing. In this situation, it can be difficult to remove a taped-together surgical drape as described above with one hand without moving the dressing with the other hand. For example, when an epidural is inserted in the spine, or arterial lines are inserted in the wrist, small drapes with a single hole are utilized. At the conclusion of the procedure, the practitioner has to hold down his/her work and rip the drape off that is attached via adhesive to the skin. This removal procedure can be quite cumbersome and cause dressings and lines to be undesirably disturbed.

The present inventor has developed a surgical drape and a method of using the surgical drape that, in some arrangements, is believed to provide certain benefits and advantages not previously available from surgical drapes known heretofore.

### SUMMARY

According to one aspect, a surgical drape is disclosed that has a sheet formed of surgical drape material. A first frangible outline is defined on the sheet and defines a first area of the sheet that is separable from the sheet by tearing along the first frangible outline to define a first fenestration through the sheet. A frangible line is defined on the sheet, wherein the frangible line extends from the first frangible outline to an outer peripheral edge of the sheet and defines a pre-defined path of tearing along which the sheet may be torn from the first area to the outer peripheral edge.

According to another aspect, a method of using a surgical drape as disclosed herein to drape and undrape a patient for a surgical procedure is disclosed.

Other aspects will become apparent upon consideration of the following detailed description.

### BRIEF DESCRIPTION OF THE DRAWINGS

FIG. 1 is top plan view of a surgical drape according to some aspects of the disclosure;

FIG. 2 is top plan view of a surgical drape according to additional aspects of the disclosure;

FIG. 3 is an isometric view of a surgical drape according to further aspects of the disclosure; and

FIG. 4 is a schematic isometric view of a surgical drape according to aspects of the disclosure in an exemplary use on a patient during a surgery.

### DETAILED DESCRIPTION

Surgical drapes according to some principles of the present of the present application are disclosed that are made of a sheet of flexible material suitable for use as a surgical drape, such as gauze, plastic, cloth, and/or paper. The surgical drapes have at least one, and preferably multiple tear-away or removable areas of different pre-defined shapes and sizes that are defined by one or more frangible outlines and surround the removable areas to allow easy removal of one or more of the removable areas by tearing along at least one of the frangible outlines. The surgical drapes also include at least one frangible path that forms a pre-defined line of easy tearing extending from the outer periphery of the sheet to an innermost one of the removable areas. A user, such as a nurse or surgeon, may select the size of an opening to be defined and surrounded by the surgical drape and remove one or more of the removable areas that corresponds with the selected size. After the surgical drape has been attached to a patient's skin, such as by tape or adhesive patches, the sheet may be easily torn along the frangible line with one hand of a user while, for example, the other hand of the user holds a dressing or needle/tubing that is inserted into or attached to the patient through the opening. In some arrangements, the surgical drapes optionally include an opening through the sheet in at least one of the removable areas, sized and arranged to facilitate easier grasping of the removable area by, for example, the user's finger, and thereby facilitate easier removal of the removable area.

Turning now to the drawings, FIG. 1 shows a surgical drape 10 embodying some principles of the present disclosure. The surgical drape 10 is formed from a sheet 12 of surgical drape material defining an outer peripheral edge 14 surrounding the sheet. The surgical drape 10 includes frangible outlines 20a, 20b, and 20c on the sheet 12, which define removable areas 22a, 22b, and 22c, respectively, for creating fenestrations of pre-defined shape and size through the sheet 12, and a frangible line 24 defined on the sheet 12, as described in further detail herein after. Optionally, the sheet may include an aperture 26 though an interior area of the sheet and adhesive 18 for attaching the sheet to the skin of a patient.

The sheet 12 may be made of any material sufficient for use as surgical drape material, including for example woven cloth, such as cotton or nylon cloth, non-woven materials, such as plastic, foil, or paper, and various combinations of the same, such as cloth laminated with paper and a water resistant layer of plastic or foil. Preferably, the sheet 12 is flexible and thin, having opposite first and second sides, such as a front side 16 and a back side (not visible), wherein the outer peripheral edge 14 extends around and connects the front and back sides of the sheet 12. The outer peripheral edge 14 may take any shape or form as desired for a selected purpose. In the exemplary embodiments shown herein, the outer peripheral edge 14 is generally rectangular or square, having opposite, spaced apart left and right edge portions 14a, 14b, which extend between opposite, spaced apart top and bottom edge portions 14c, 14d. (Directional modifiers, such as left, right, top, bottom, front, back, etc., are used herein for ease of reference with respect to the various views shown in the drawings and are not intended as limitations on the exact arrangement of the structures modified thereby.) The surgical drape 10 is not limited to the specific shape disclosed, however, and in other embodiments the outer peripheral edge 14 may be, for example, circular, oval, polygonal, or any other regular or irregular shape as desired or as may be useful for a particular application.

At least one, and preferably two, three, or more frangible outlines are formed on the sheet 12 to define various pre-defined areas of tear away portions of the sheet that may be easily separated from the sheet 12 by simply tearing along a selected one of the frangible outlines. The first frangible outline 20a, preferably in the shape of an enclosed outline, such as a circle, oval, rectangle, etc., is located near a central region of the sheet 12. The frangible outline 20a is spaced inwardly from the outer peripheral edge 14 and defines the first removable area 22a of the sheet 12 that is separable from the sheet by tearing the area 22a along the first frangible outline 20a. Thus, when the sheet 12 is torn along the frangible outline 20a, the first area 22a is removed from the sheet 12, which leaves a first hole or opening (also known as a fenestration) through the sheet 12 at a location, size, and shape that is pre-defined by the frangible outline 22a. The shape of the first frangible outline 20a and size of the first area 22a may be in any shape and area as desired for a particular use and/or application. In the exemplary embodiment shown in FIG. 1, the first frangible outline 20a is in the form of a circle radially aligned at a center point of the sheet 12, however other locations on the sheet and other shapes are clearly possible within the scope of the disclosure.

The surgical drape 10 preferably includes one or more additional removable areas, such as the removable areas 22b and 22c defined by the second and third frangible outlines 20b and 20c, respectively. In the depicted arrangement, the second frangible outline 20b is spaced radially outwardly from the first frangible outline 20c and radially inwardly from the outer peripheral edge 14, and the third frangible outline 20c is spaced radially outwardly from the second frangible outline 20b and radially inwardly from the outer peripheral edge 14. The second frangible outline 20b entirely surrounds, such as by encircling, the first frangible outline 20a, and the third frangible outline 20c entirely surrounds, such as by encircling, the second frangible outline 20b. Correspondingly, the second frangible outline 20b defines a second removable area 22b surrounding the first removable area 22a, and the third frangible outline 20c defines a third removable area 22c that surrounds the second removable area 22b. In one exemplary arrangement, each of the first, second, and third frangible outlines 20a-20c is circular and concentrically aligned about a center point of the sheet 12. The first frangible outline 20a has a diameter of approximately two inches, the second frangible 20e has a diameter of approximately four inches, the third frangible outline 20c has a diameter of approximately six inches, and the sheet 12 has a size of approximately ten inches by ten inches square; however, the disclosure is not limited to the exemplary dimensions provided herein and other dimensions may be provided as desired for a particular intended use. With the multiple frangible outlines 20a-20c, the user may selectively form a fenestration through the sheet 12 of various pre-defined sizes corresponding with the first removable area 22a, the first and second removable areas 22a and 22b, or the first, second, and third removable areas 22a-22c, depending on which of the removable areas 22a-22c are removed from the sheet 12 by tearing along one of the frangible outlines 20a, 20b or 20c. For example, the first and second removable areas 22a-b are separable from the sheet 12 by tearing along the second frangible outline 20b to define a second fenestration through the sheet that is larger than the fenestration formed by tearing along the first frangible outline 20a. Similarly, an even larger third fenestration may be formed through the sheet 12 by removing the first, second, and third removable areas 22a-c by tearing along the third frangible outline 20c. The surgical drape 10 is not limited to having any particular number of frangible outlines 20a-20c and corresponding removable areas 22a-22c, and may include more or fewer than the three frangible outlines 20a-c shown and described herein, which may provide even more flexibility of use with different surgical procedures. Preferably, the sheet 12 has an outer peripheral area 22d defined between the outermost of the frangible outlines, such as the third frangible outline 22c as shown in FIG. 1, and the outer peripheral edge 14. The outer peripheral area 22d preferably surrounds each of the removable areas 22a-c, whereby when any or all of the removable areas 22a-c are removed from the sheet 12, the surgical drape 10 still has the form of a sheet surrounding a fenestration through a central portion of the sheet.

The frangible line 24 forms a pre-defined tear path on the sheet 12 that extends from the outer peripheral edge 14 at least to the first frangible outline 20a, and preferably extends from the outer peripheral edge 14 completely to any of the fenestrations that can be formed through the sheet 12 by tearing along any one of the frangible outlines 20a-c. The frangible line 24 may be straight as shown, for example, in FIG. 1. However, the shape of the frangible 24 line is not so limited and may be arcuate, wavy, have polygonal segments, etc., as long as it extends along a pre-defined path from or near the outer peripheral edge 14 at least to the frangible outline 20a. The frangible line 24 defines a path along which the sheet 12 may be easily torn from the first area 22a to the outer peripheral edge 14. The frangible line 24 and the frangible outlines 20a-c described herein may be any formation that defines a path along which the sheet 12 will tear more easily than surrounding areas, such as perforations extending entirely through the sheet 12, thinned sections of the sheet 12 in relation to the adjacent portions of the sheet, dye crimped portions of the sheet, etc., as long as the frangible line or outline provides an area of weakness which will direct tearing of the sheet 12 along a pre-defined path in a manner well understood in the art. In a preferred arrangement, the frangible line 24 extends from the outer peripheral edge 14 all the way to the edge of the aperture 26, thereby facilitating an easy and predictable pre-defined tear line along the sheet 12 from the outer peripheral edge 14 to any one of the frangible outlines 20a-20c and to the aperture 26, if desired. Further, the frangible line 24 is shown in the exemplary embodiment of FIG. 1 to be a radial line extending from the center of the concentrically aligned frangible outlines 20a-20c; however the frangible line 24 is not limited to being radially aligned and may be radially offset and/or even tangentially aligned with one or more of the aperture 26 and/or the first through third frangible outlines 20a-20c. Optionally, the surgical drape 10 may include additional frangible lines that provide additional pre-defined paths of tearing from the outer peripheral edge 14 to interior portions of the sheet 12. For example, an optional second frangible line 24a may be formed in the sheet 12 extending from the first area 22a to the left peripheral edge 14a, such as extending radially outwardly from the aperture 26 and in alignment with the frangible line 24, which may provide a complete tear-away path from opposite peripheral edges 14a to 14b through any fenestration formed with any of the frangible outlines 22a-c.

The surgical drape 10 preferably includes an opening, such as the aperture 26, through a central area to facilitate gripping by the user. The aperture 26 extends through the first removable area 22 of the sheet 12 and is spaced radially inwardly from the first frangible outline 20a. The aperture 26 preferably has a size sufficient to allow a users finger to extend through the sheet 12. In some arrangements, the aperture 26 may be useful for facilitating removal of one or more of the first, second, and third removable areas 22a-22c by allowing a user to extend a finger through the sheet and grasp the sheet on the opposite front side 16 and back side between the users fingers to selectively tear one of the first, second, and/or third areas from the sheet 12 along the corresponding first, second, or third frangible outline 20a-20c. In one exemplary embodiment, the aperture 26 is a circular opening with a diameter of approximately one half inch; however, the disclosure is not limited to this exemplary dimension and other suitable shapes and dimensions may also be utilized. Further, the aperture 26 preferably is concentrically aligned with the circular frangible outlines 20a-20c at the center of the sheet 12; however, the aperture 26 however may take other sizes and shapes and is not limited to the exemplary size and shape shown in the drawings.

In one preferred arrangement, a notch 28 is formed through the sheet 12 located at one or both ends of the frangible line 24. In the exemplary embodiment shown in FIG. 1, the notch 28 is disposed across the frangible line 24 at the outer peripheral edge 14. Optionally, the notch 28 may be formed across the frangible line 24 along the edge of the aperture 26, either additionally or alternatively to forming the notch 28 along the peripheral edge 14. The notch 28 may take any shape or form sufficient to be useful to facilitate readily starting a tear along the frangible line 24 and thereby facilitate easily and selectively tearing the sheet 12 along the frangible line 24. For example, the notch 28 may be triangularly shaped, as shown in the figure, or the notch may take other shapes, such as arcuate, polygonal, or linear.

The adhesive 18 is optionally disposed on a side of the sheet 12 to facilitate easily securing the surgical drape 10 to the skin of a patient. Preferably, the adhesive is disposed on only one side, such as the back side, as shown in FIG. 1. The adhesive 18 preferably is disposed on at least the outer peripheral portion 22d of the sheet 12 partly or completely surrounding the outermost frangible outline 22c sufficient to adhere the surgical drape 10 to a patient when any or all of the removable areas 22a-c are removed. In the depicted arrangement, the adhesive 18 is disposed on each of the removable areas 22a-c and the outer peripheral area 22d, and preferably covers substantially the entire back side of the sheet 12 with a space or gap between the adhesive 18 and each of the outer peripheral edge 14 and the edge of the aperture 26. The adhesive 18 is preferably a bio-compatible adhesive appropriate for removably securing the sheet 12 to a patient's skin. The surgical drape may be secured to the patient's skin in other manners, such as with adhesive tape or bio-compatible glue, in addition or alternatively to the adhesive 18. In a preferred arrangement, the adhesive 18 is covered by a removable backing tape during storage, which backing tape is subsequently removed to expose the adhesive when the user desires to attach the surgical drape 10 to a patient.

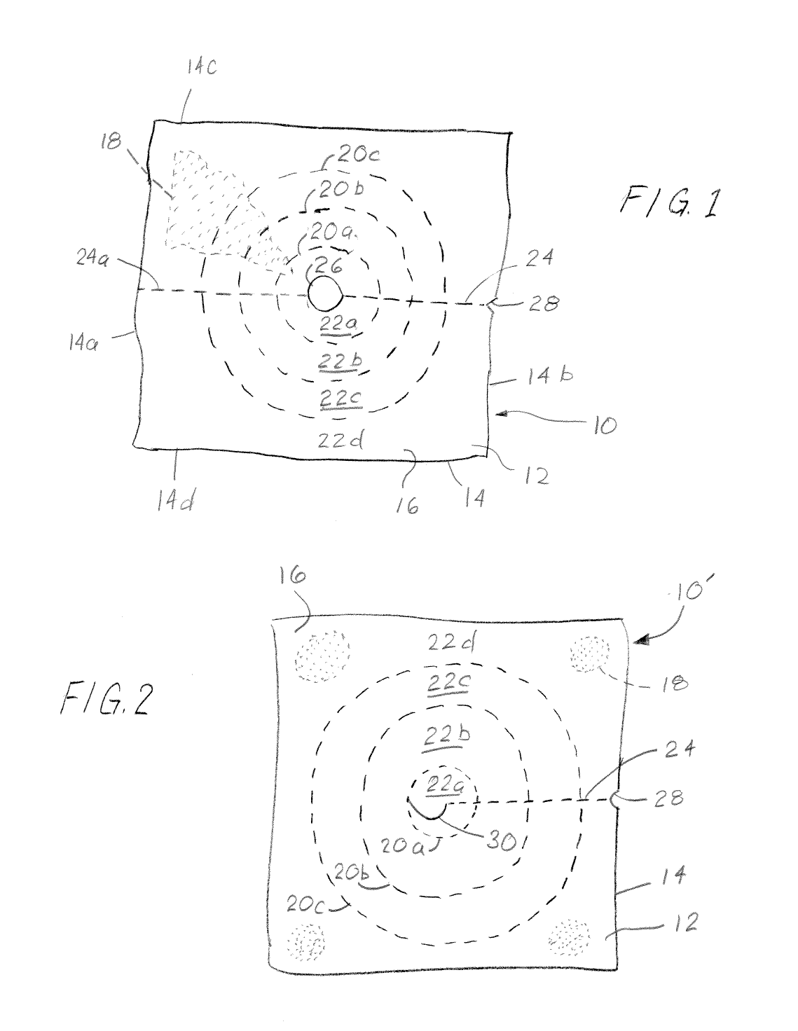

Turning to FIG. 2, another surgical drape 10′ is shown that exemplifies additional aspects of the disclosure. Similar to the surgical drape 10, the surgical drape 10′ includes a sheet 12 of surgical drape material defining an outer peripheral edge 14 and having first, second, and third frangible outlines 20a, 20b, 20c defining first, second, and third removable areas 20a, 20b, 20c, and a frangible line 24 extending from the outer peripheral edge 14 to an opening in the first removable area 20a, all substantially as described previously herein. However, in the surgical drape 10′, the opening has the form of a slit 30 extending through the sheet 12 instead in the form of the aperture 26, and the adhesive 18 is formed as dots or circular areas disposed on the outer peripheral area 22d of the sheet 12. The slit 30 forms a pre-defined opening that is preferably arranged to allow a user to extend a finger through the slit 30 to facilitate easily gripping and tearing the removable area of the first removable area 22a as substantially as described previously. The slit may take any shape sufficient to allow the insertion of a finger such, as a straight line, an X, an arcuate line, etc. In the exemplary arrangement shown in FIG. 2, the slit 30 has an arcuate shape, such as a circular arc section spanning 90 degrees. The slit 30 is preferably spaced radially inwardly from the first frangible outline 20a; however, in some arrangements the slit 30 extends radially outwardly to engage the first frangible outline 20a. Preferably, the slit 30 also engages, such as by intersecting with, the frangible line 24. The slit 30 may be formed by any convenient means, such as by die cutting. The slit 30 is different from the aperture 26, in that the slit 30 has an elongate linear or arcuate dimension and does not have a substantial width dimension transverse to the elongate dimension. Remaining portions of the surgical drape 10′ are substantially similar as previously described herein with regard to the surgical drape 10.

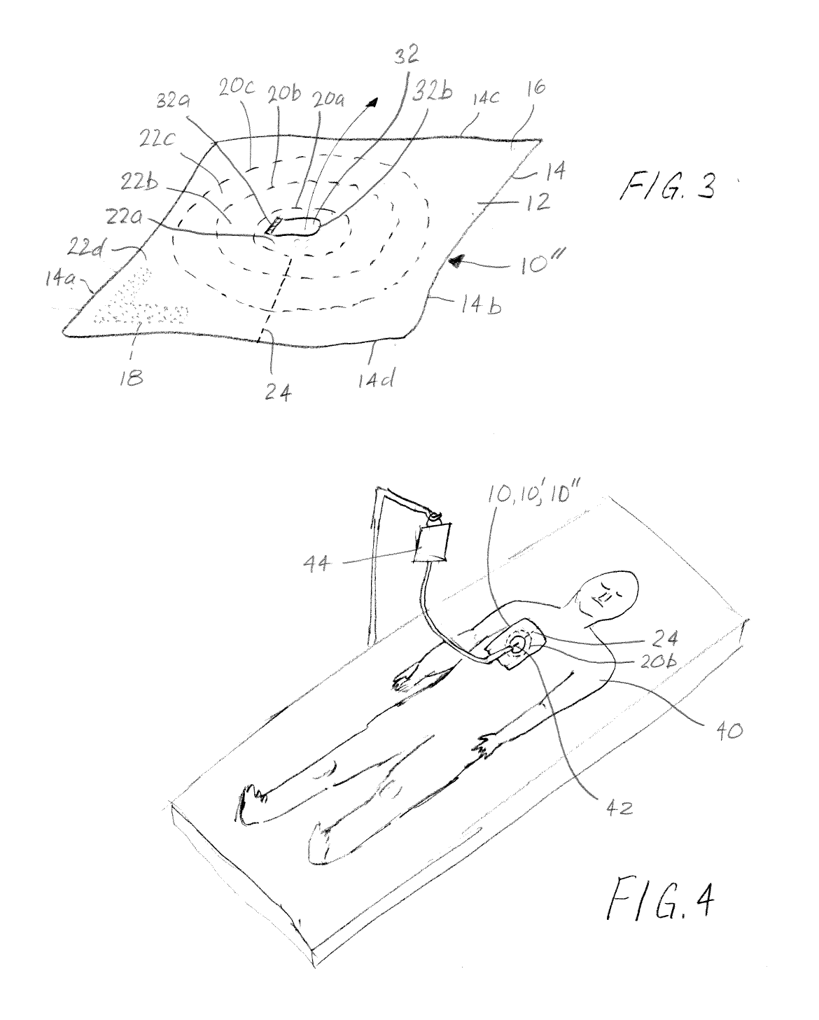

In FIG. 3, a further surgical drape 10″ exemplifies additional features according to the principles of the present disclosure. The surgical drape 10″ includes a sheet 12, first, second, and third frangible outlines 20a-20c, and a frangible line 24, each substantially as shown and described previously herein above except as noted hereinafter. One difference is a tab 32 that extends outwardly from the front side 16, preferably extending from the first removable area 22a. The tab 32 may in some arrangements facilitate easy removal of at least the first removable area 22a by providing a structure for a user to grip easily with one hand while tearing one of the first, second, and/or third frangible outlines 20a-20c. The tab 32 is formed integrally with the sheet 12 or is formed separately and subsequently secured to the sheet 12 by any sufficient fasteners, such as adhesive, stitching, or welding. In a preferred arrangement, a first end 32a of the tab 32 is attached to the sheet 12 in the first removable area 22b, and a second end 32b, which is opposite the first end 32a, is unattached to the sheet 12, thereby defining a free end of the tab which a user may lift from the sheet and grasp during the process of tearing and removing any of the removable areas 22a-22c. The surgical drape 10″ optionally includes openings, such as the aperture 26 and/or the slit 30, to provide one or more additional mechanisms to facilitate removal of one or more of the various removable areas 22a-22c. In the exemplary embodiment shown in FIG. 3, there is no opening such as the aperture 26 or 30 arranged through the sheet, and the frangible line 24 extends from the outer peripheral edge 14 to the first frangible outline 20a, i.e. the innermost frangible outline defining the inner most removable area, and terminates at the first frangible outline 20a. Another difference is that the adhesive 18 is disposed in an area extending along the outer peripheral edge 14, such as a linear strip of adhesive spaced inwardly from the outer peripheral edge and surrounding partly or completely the outermost frangible outline, such as the frangible outline 20c. Remaining portions and features of the surgical drape 10″ shown are substantially similar to the same features as shown and described previously herein.

FIG. 4 shows one exemplary use of a surgical drape 10, 10′, 10″ as shown and described herein in use on a patient 40 during a surgical procedure. In a first step, the user, such as a doctor or nurse, selects one of the pre-determined sizes of fenestrations to make through the surgical drape 12. Thereafter, the user forms the selected fenestration through the surgical drape 10, 10′, 10″ by tearing along a selected one of the frangible outlines 20a-20c and removing the removable area or areas 22a-c that are surrounded by the selected frangible outline from the sheet 12. Preferably, as described above, the selected frangible outline is spaced from the outer peripheral edge 14 of the sheet so that at least an outer peripheral area 22d of the sheet remains for placement on the patient 40. The surgical drape 10, 10′, 10″ is attached to the patient 40 with the fenestration aligned over a region of interest, such as a surgical area or area where a needle 42, such as from a syringe or a drug supply 44, is to be inserted into the patient 40 such that the fenestration exposes the region of interest and the outer peripheral area 22d of the sheet thereby surrounds the region of interest. The fenestration may be formed through the sheet 12 either before or after the surgical drape 10, 10′, 10″ is attached to the patient 40. The surgical drape is attached to the patient 40 with adhesive tape and/or with the adhesive 18 disposed on the sheet 12. Various surgical procedures may be performed on the patient in the region of interest, such as inserting the needle 42 into the patient through the fenestration. When it is desired to remove the surgical drape 10, 10′, 10″ from the patient 40, a user optionally tears the sheet 12 along the frangible line 24, such as by starting at the notch 28, from the outer peripheral edge 14 to the fenestration or vice versa and removes the sheet 12 from the patient. In this manner, for example, the surgical drape 10, 10′, 10″ can be removed from the patient and from around the needle 42 while the needle is still inserted in the patient without having to use scissors to cut the sheet 12.

### INDUSTRIAL APPLICABILITY

In some aspects, the surgical drapes disclosed herein are useful for covering portions of a patient surrounding an incision or other penetration through the skin. However, the surgical drapes described herein are not limited to any particular exemplary uses and procedures detailed herein and may be used in any other manners or application as desired and would be understood by one of ordinary skill in the art.

Numerous modifications to the surgical drapes disclosed herein will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications within the scope of the appended claims are reserved.

I claim:

1. A surgical drape comprising:
a sheet formed of surgical drape material and having an outer peripheral edge;
a first frangible outline defined on the sheet, wherein the first frangible outline is spaced inwardly from the outer peripheral edge and defines a first area of the sheet that is separable from the sheet by tearing along the first frangible outline to define a first fenestration through the sheet; and
a frangible line defined on the sheet, wherein the frangible line extends from the first frangible outline to the outer peripheral edge and defines a pre-defined path of tearing along which the sheet may be torn from the first area to the outer peripheral edge.

2. The surgical drape of claim 1, further comprising:
an opening through the sheet, wherein the opening is located in the first area.

3. The surgical drape of claim 2, wherein the opening comprises an elongate slit through the sheet.

4. The surgical drape of claim 3, wherein the slit engages the first frangible outline.

5. The surgical drape of claim 4, wherein the slit engages the frangible line, and wherein the slit is arcuate.

6. The surgical drape of claim 2, wherein the opening comprises an aperture.

7. The surgical drape of claim 6, wherein the aperture is spaced radially inwardly from the first frangible outline, and wherein the aperture is sized sufficiently to receive a finger.

8. The surgical drape of claim 7, wherein the aperture is circular and concentric with the first frangible outline.

9. The surgical drape of claim 1, further comprising:
a second frangible outline defined on the sheet, wherein the second frangible outline surrounds the first area and is spaced between the outer peripheral edge and the first frangible outline, and wherein the second frangible outline defines a second area of the sheet that is separable from the sheet by tearing along the second frangible outline to define a second fenestration through the sheet that is larger than the first fenestration.

10. The surgical drape of claim 9, wherein the first frangible outline defines a first circle, the second frangible outline defines a second circle, and the first and second circles are concentric.

11. The surgical drape of claim 10, wherein the frangible line extends radially in a straight line from the first frangible outline to the outer peripheral edge.

12. The surgical drape of claim 1, further comprising:
a tab extending from the first area of the sheet, wherein the tab has a first end attached to the first area of the sheet and a second end not attached to the sheet.

13. The surgical drape of claim 1, wherein the sheet is flexible, and wherein the surgical drape material comprises gauze.

14. The surgical drape of claim 1, further comprising:
a layer of adhesive on a side of the sheet and disposed adjacent the outer peripheral edge.

15. The surgical drape of claim 1, further comprising:
a notch through the sheet, the notch disposed along the outer peripheral edge and in engagement with the frangible line.

16. A method of draping and undraping a patient for a surgical procedure, the method comprising the steps:
forming a fenestration through a surgical drape comprising a sheet of surgical drape material by tearing an inner area of the sheet along a first frangible outline spaced from an outer peripheral edge of the sheet and removing the area;
attaching the surgical drape to the patient with the fenestration aligned over a region of interest and thereby exposing the area of interest through the fenestration;
tearing the attached sheet along a frangible line in the sheet extending from the fenestration to the outer peripheral edge; and
removing the torn sheet from the patient.
